(19) United States
(12) Patent Application Publication
(10) Pub. No.: US 2008/0234785 A1
(43) Pub. Date: Sep. 25, 2008

(54) SLEEP CONTROLLING APPARATUS AND METHOD, AND COMPUTER PROGRAM PRODUCT THEREOF

(75) Inventors: Kanako Nakayama, Kanagawa (JP); Takuji Suzuki, Kanagawa (JP); Akihisa Moriya, Tokyo (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Kabushiki Kaisha Toshiba, Tokyo (JP)

(21) Appl. No.: 11/854,980

(22) Filed: Sep. 13, 2007

(30) Foreign Application Priority Data
Mar. 23, 2007 (JP) 2007-077072

(51) Int. Cl. A61N 1/08

(52) U.S. Cl. 607/62

(57) ABSTRACT

A sleep controlling apparatus includes a measuring unit that measures biological information of a subject; a first detecting unit that detects a sleeping state of the subject selected from the group consisting of a falling asleep state, a REM sleep state, a light non-REM sleep state and a deep non-REM sleep state, based on the biological information measured by the measuring unit; a first stimulating unit that applies a first stimulus of an intensity lower than a predetermined threshold value to the subject when the light non-REM sleep state is detected by the first detecting unit; and a second stimulating unit that applies a second stimulus of an intensity higher than the first stimulus after the first stimulus is applied to the subject.

### CROSS-REFERENCE TO RELATED APPLICATIONS

This application is based upon and claims the benefit of priority from the prior Japanese Patent Application No. 2007-77072, filed on Mar. 23, 2007; the entire contents of which are incorporated herein by reference.

### BACKGROUND OF THE INVENTION

1. Field of the Invention

The present invention relates to a sleep controlling apparatus and method, and computer program product for controlling sleep conditions of a subject.

2. Description of the Related Art

Researches on sleep controlling apparatuses have been conducted for the purpose of improving a refreshed feeling at awakening, which is an important factor in sleep. In comparison with an alarm clock that is widely used, conditions of easily waking up in biological rhythms are taken into consideration to design the sleep controlling apparatuses. Such sleep controlling apparatuses are receiving attention because of their capability of waking people up with a refreshed feeling.

Among these sleep controlling apparatuses, apparatuses that focus on a circadian rhythm and a sleep rhythm of biological rhythms are known. For instance, there is a type of apparatus that induces a light sleep state in the sleep rhythm and leads the circadian rhythm to an active phase. An apparatus of another type measures the sleep rhythm of a subject and induces arousal when the subject is in a state of easily waking up.

JP-A 07-318670 (KOKAI), for example, discloses a technology of irradiating the subject with light at the subject's desired rising time. The circadian rhythm is led to an active phase by gradually increasing the irradiation light intensity in three levels. In addition, JP-A 2006-43304(KOKAI) discloses a technology of controlling the sleep rhythm to bring the subject to a state of easily waking up at a desired rising time. More specifically, those technologies measure the sleep states and thereby make projections on the sleep state at the time of rising from the sleep rhythm after falling asleep. If the estimated depth of sleep does not reach a predetermined depth, a stimulus is given to the body during the sleep. The conventional technologies thereby bring the sleep to a depth with which the subject can easily wake up at a desired time of rising. Moreover, Akihisa Moriya et al. teach a technology of detecting the REM sleep state in real time and sounding a wake-up alarm during the REM sleep in “REM Sleep Detection by Autonomic Nervous Analysis and Application thereof” (Proceedings of the 19th Annual Symposium on Biological and Physiological Engineering, (Osaka), November 2004, pp. 207-208).

The above mentioned JP-A 07-318670 (KOKAI), however, does not suggest measurement of the biological rhythm, and thus the timing of the wake-up stimulus depends on the desired time of rising. This conventional technology cannot cope with individual differences. Furthermore, the technology may interfere with the sleep rhythm and thus may not always be an effective method. The technology of JP-A 2006-43304(KOKAI) may interfere with deep sleep, and thus may prevent a subject from having sufficient deep sleep.

### SUMMARY OF THE INVENTION

According to one aspect of the present invention, a sleep controlling apparatus includes a measuring unit that measures biological information of a subject; a first detecting unit that detects a sleeping state of the subject selected from a group consisting of a falling asleep state, a REM sleep state, a light non-REM sleep state and a deep non-REM sleep state, based on the biological information; a first stimulating unit that applies a first stimulus of an intensity lower than a predetermined threshold value to the subject when the light non-REM sleep state is detected; and a second stimulating unit that applies a second stimulus of an intensity higher than the first stimulus after the first stimulus is applied to the subject.

According to another aspect of the present invention, a sleep controlling method includes measuring biological information of a subject; detecting a sleeping state of the subject selected from the group consisting of a falling asleep state, a REM sleep state, a light non-REM sleep state and a deep non-REM sleep state, based on the biological information; applying a first stimulus of an intensity lower than a predetermined threshold value to the subject when the light non-REM sleep state is detected; and applying a second stimulus of an intensity higher than the first stimulus after the first stimulus is applied to the subject.

A computer program product according to still another aspect of the present invention causes a computer to perform the method according to the present invention.

### BRIEF DESCRIPTION OF THE DRAWINGS

FIG. 1 is a block diagram illustrating the entire structure of a sleep controlling system according to a first embodiment;

FIG. 2 is a schematic perspective diagram illustrating an example of a subject wearing the sleep controlling system illustrated in FIG. 1;

FIG. 3 is an explanatory diagram illustrating the process of an autonomic-nerve index calculating unit;

FIG. 4 is an explanatory diagram illustrating the process of a cycle frame setting unit;

FIG. 5 is a diagram illustrating autonomic nerve indices obtained during the sleep;

FIG. 6 is a schematic diagram illustrating a circadian rhythm;

FIG. 7 is a schematic diagram for explaining the relationship between a sleep state and a body temperature in a deep portion;

FIG. 8 is a flowchart of a sleep controlling process performed by the sleep controlling system;

FIG. 9 is a flowchart of a sleep state determining process;

FIG. 10 is a schematic diagram illustrating a hardware structure of the main body according to the embodiment;

FIG. 11 is a flowchart of a sleep controlling process performed by a sleep controlling system according to a second embodiment;

FIG. 12 is a flowchart of a sleep controlling process performed by a sleep controlling system according to a third embodiment;

FIG. 13 is a flowchart of an effectiveness checking process;

FIG. 14 is a flowchart of an effectiveness checking process on a sleep controlling system according to a fourth embodiment;

FIG. 15 is a flowchart of an effectiveness checking process on a sleep controlling system according to a fifth embodiment;

FIG. 16 is a flowchart of a sleep controlling process on a sleep controlling system according to a sixth embodiment;

FIG. 17 is a block diagram illustrating the entire structure of a sleep controlling system according to a seventh embodiment;

FIG. 18 is a flowchart of a sleep controlling process on the sleep controlling system according to the seventh embodiment;

FIG. 19 is a flowchart of a nighttime sleep controlling process; and

FIG. 20 is a flowchart of a daytime sleep controlling process.

### DETAILED DESCRIPTION OF THE INVENTION

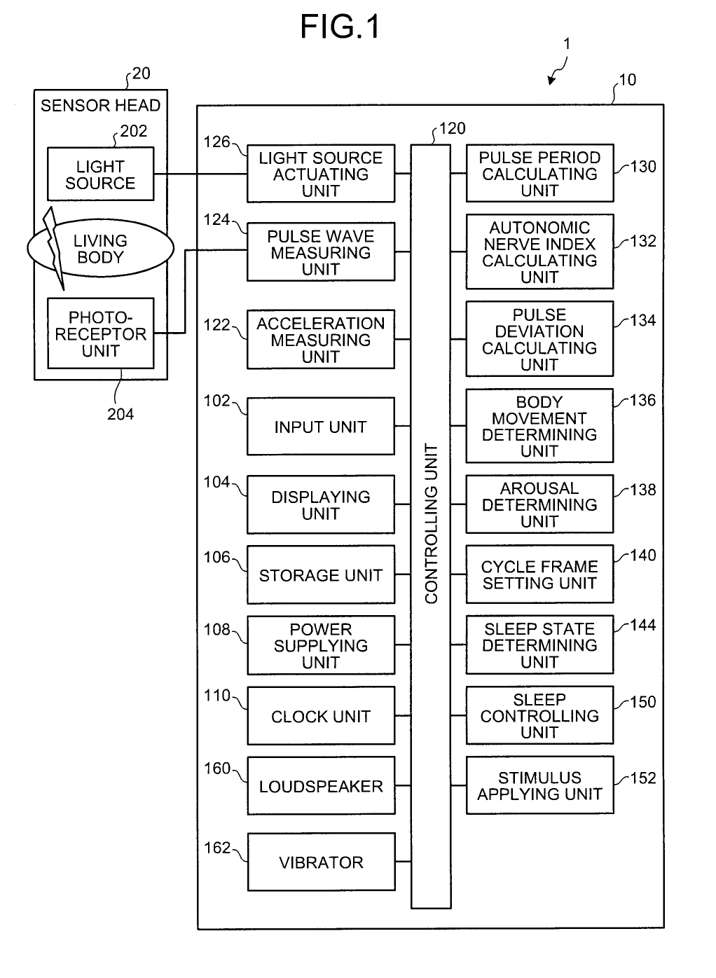

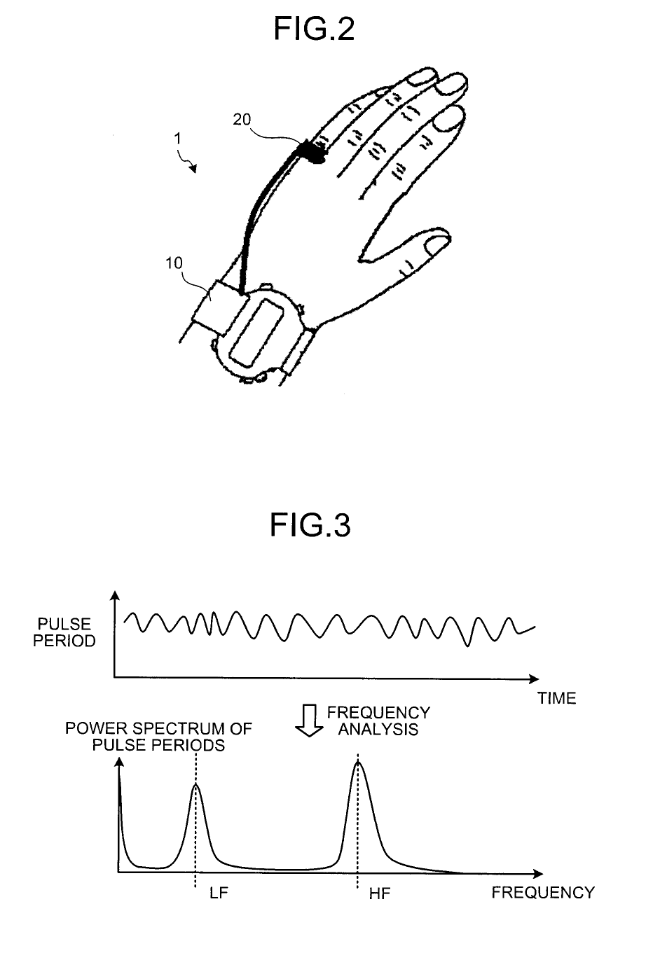

A sleep controlling system 1 includes a system main body 10 and a sensor head 20 for pulse wave measurement, as illustrated in FIG. 1. The main body 10 may be worn in a manner similar to a wrist watch around the wrist, as indicated in FIG. 2. The sensor head 20 has a shape of a ring as illustrated in FIG. 2, to be worn on the little finger. The main body 10 detects a change in the sleep state based on the result of the measurement conducted by the sensor head 20, and gives a stimulus to a subject in accordance with the change so that the subject can wake up refreshed. The shape of the sensor head 20 is not limited to a ring, but the sensor head 20 may be configured into a shape of a mat.

The main body 10 includes an input unit 102, a displaying unit 104, a storage unit 106, a power supplying unit 108, a clock unit 110, a controlling unit 120, an acceleration measuring unit 122, a pulse wave measuring unit 124, a light source actuating unit 126, a pulse period calculating unit 130, an autonomic-nerve index calculating unit 132, a pulse deviation calculating unit 134, a body movement determining unit 136, an arousal determining unit 138, a cycle frame setting unit 140, a sleep state determining unit 144, a sleep controlling unit 150, and a stimulus applying unit 152.

The input unit 102 receives from a subject an instruction for turning on/off the power of the sleep controlling system 1. In addition, the input unit 102 receives a desired time of rising, desired hours of sleep, and the like. The input unit 102 may be a switch or the like. The displaying unit 104 is a displaying device that displays results of sleep state determination made by the main body 10. The storage unit 106 is a recording medium such as a memory that stores therein measurement data including pulse wave data and body movement data, processed data, the desired time of rising and the desired hours of sleep input by the subject on the input unit 102, and the like.

The power supplying unit 108 supplies power to the sleep controlling system 1, and in particular, it is a battery or the like. The clock unit 110 keeps times of day, and in particular, it is a real-time clock IC or the like.

The controlling unit 120 controls timings of measurement, stores and processes the received data. The acceleration measuring unit 122 is an acceleration sensor that measures acceleration data as body movement data that indicates an amount of body movement of the subject, and performs data conversion. In particular, this acceleration sensor measures the acceleration of −2 to 2 Gs in three axial directions. The acceleration measuring unit 122 has a regulator circuit that regulates the gain and offset of analog data obtained by the acceleration sensor, and converts the regulated data into a digitized amount by a 10-bit A/D converter. Then, the acceleration measuring unit 122 outputs the converted data to the controlling unit 120.

The sensor head 20 includes a green LED, which serves as a light source 202, and a photodiode, which serves as a photoreceptor unit 204. The sensor head 20 irradiates the surface of the skin with light, and captures, by use of the photodiode, changes to the reflected light caused by changes in the bloodstream of the capillaries.

The pulse wave measuring unit 124 measures the pulse data of the subject and converts the data. The pulse wave measuring unit 124 converts the output current from the photodiode that serves as the photoreceptor unit 204 into a voltage by use of a current-voltage converter, and amplifies the voltage by use of an amplifier. After a high-pass filter (cutoff frequency: 0.1 hertz) and a low-pass filter (cutoff frequency: 50 hertz) are applied thereto, the pulse wave measuring unit 124 converts the voltage into a digitized amount by use of the 10-bit A/D converter. Thereafter, the pulse wave measuring unit 124 outputs the converted pulse wave data to the controlling unit 120. The light source actuating unit 126 is a voltage supplying unit that actuates the light source 202.

The pulse period calculating unit 130 calculates a pulse period from the pulse wave data obtained by the pulse wave measuring unit 124 and thereby generates pulse period data. A pulse period means a period of time for a cycle of a pulse wave. More specifically, the pulse period calculating unit 130 samples a string of pulse wave data from the pulse wave measured by the pulse wave measuring unit 124. Then, the pulse period calculating unit 130 conducts time differentiation on the sampled string of pulse wave data to acquire direct current variant components of the string of pulse wave data. Further, the pulse period calculating unit 130 removes direct current variant components from the string of plus wave data.

Then, the pulse period calculating unit 130 acquires the maximum and minimum values for pulse wave data having a length of approximately one second around the processed point of the string of pulse wave data from which the direct current variant components have been removed. A value between the maximum and minimum values is set to a pulse wave period threshold value. As a pulse wave period threshold value, it is preferable to take on a value at 90 percent of the amplitude with respect to the minimum value, where the amplitude denotes a difference between the maximum and minimum values. Then, the pulse period calculating unit 130 calculates the times at which a value of the pulse wave data that corresponds to this pulse wave period threshold value appears among the series of pulse wave data from which the direct current variant component has been removed, and determines the interval between the calculated times as a pulse period (pulse period data).

The pulse period data has irregular intervals. The pulse period data having irregular intervals need to be converted to data having regular intervals to conduct a frequency analysis. The pulse period calculating unit 130 therefore interpolates and re-samples the pulse period data of irregular intervals to generate pulse period data of regular intervals. For instance, the pulse period calculating unit 130 generates pulse period data of regular intervals in accordance with the cubic interpolation, where three sampling points including the interpolated point and before and after this point are used.

The autonomic-nerve index calculating unit 132 finds two autonomic nerve indices, the index LF for a low frequency region (in the vicinity of 0.05 to 0.15 hertz) and the index HF for a high frequency region (in the vicinity of 0.15 to 0.4 hertz) to determine the sleep state, and defines the calculated data as autonomic nerve index data. More specifically, the autonomic-nerve index calculating unit 132 first converts the regular-interval pulse period data to a frequency spectrum distribution by, for example, performing a fast Fourier transform (FFT), as indicated in FIG. 3. Next, the autonomic-nerve index calculating unit 132 finds the LF and HF from the acquired frequency spectrum distribution. In particular, the autonomic-nerve index calculating unit 132 picks up the peak value of the multiple power spectra and the points before and after the peak value at the same intervals therefrom and calculates the arithmetic average of the three points, thereby obtaining the LF and HF.

According to the embodiment, the structure is configured to employ the FFT as a frequency analysis method from a standpoint of reducing the load of data processing. However, the structure is not limited thereto. Other examples of frequency analysis methods include the AR model method, maximum entropy method, and wavelet method.

The pulse deviation calculating unit 134 calculates a deviation of instantaneous pulse, or the pulse wave deviation, within, for example, a minute of the pulse wave data obtained by the pulse wave measuring unit 124. The body movement determining unit 136 obtains differential coefficients of the accelerations in the three axial directions by time-differentiating the acceleration data for the three directions obtained from the acceleration measuring unit 122. Then, the body movement determining unit 136 finds a body movement amount from the average of the variation amount of body movement data that is obtained from the square root of the sum of squares of the differential coefficients for the accelerations of the three axial directions and the variation amount of body movement data that is obtained from the average variation amount of body movement within a pulse period. The body movement determining unit 136 determines that there is a body movement when the variation amount in the body movement amount is larger than a predetermined threshold value. For instance, the body movement determining unit 136 employs 0.01 Gs, which is the minimum value of subtle movement that is used in an actigraph, as the predetermined threshold value.

The arousal determining unit 138 receives from the body movement determining unit 136 information as to whether there is any body movement, and measures the frequency of body movements in a setup section. The setup section may be preferably determined as one minute. Then, the arousal determining unit 138 determines that the subject is in an arousal state when the frequency of body movements is equal to or larger than the predetermined threshold value. On the other hand, the arousal determining unit 138 determines that the subject is in a sleep state when the frequency of body movements is lower than the predetermined threshold value. The threshold value may be determined as 20 times/minute, for example, based on the frequency of body movement during the arousal state in the past.

The cycle frame setting unit 140 sets up cycle frames. A cycle frame is a time frame that contains one cycle of sleep. One cycle of sleep is approximately between 90 and 120 minutes, and the cycle frame can be set up freely in this range. For instance, the cycle frame setting unit 140 may set up a cycle frame to a time frame of the current time to the past time 120 minutes before at the maximum.

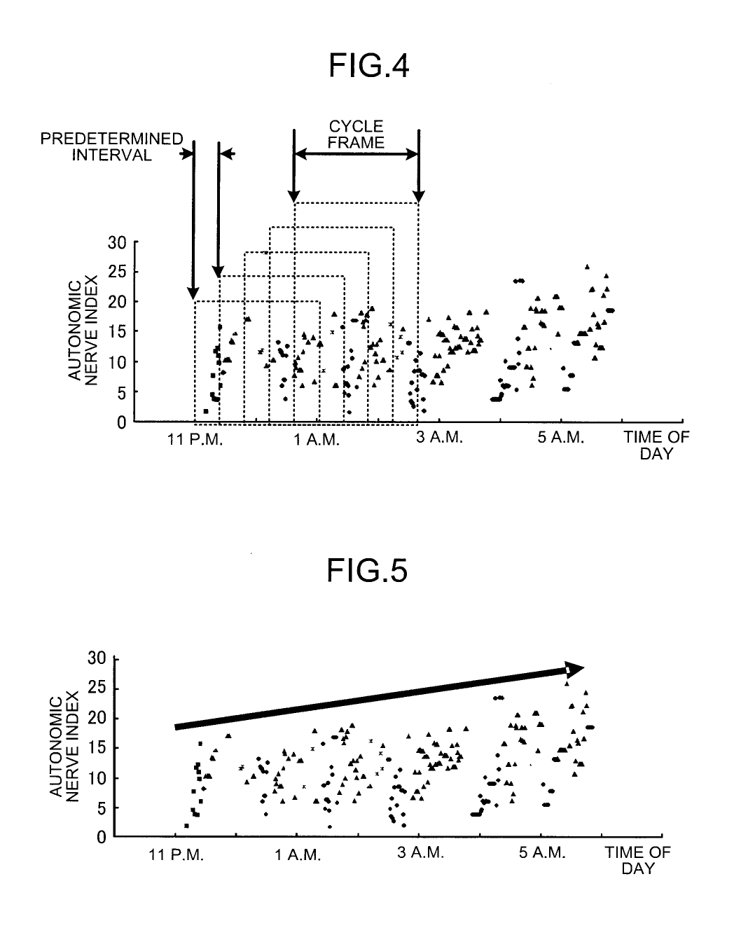

The cycle frame in the example illustrated in FIG. 4 is set to 120 minutes. It is assumed that the autonomic-nerve index calculating unit 132 starts the measurement of autonomic nerve indices at 11 p.m. In this example, the measurement of autonomic nerve indices continues to be performed after 11 p.m. At 1 a.m., because 120 minutes elapse from 11 p.m., the cycle frame setting unit 140 sets up this time frame as a cycle frame. At one minute after 1 a.m., the cycle frame setting unit 140 sets up another cycle frame for the 120 minutes between 11:01 p.m. and 1:01 a.m. In this manner, the cycle frame setting unit 140 sets up cycle frames at predetermined intervals such as for every minute. The interval should be set to a length of time that is shorter than the cycle frame. Furthermore, it is preferable that the interval be set to a length of time shorter than a continuous judgment frame, which will be described later.

The sleep state determining unit 144 determines whether the subject is in a sleep state from the activeness of autonomic nerve, based on the autonomic nerve indices LF and HF calculated by the autonomic-nerve index calculating unit 132 and the pulse deviation calculated by the pulse deviation calculating unit 134. The sleep state determining unit 144 determines the depth of sleep as the sleep state. The depth of sleep indicates how active the brain of the subject is. According to the embodiment, the depth of sleep is categorized into three levels, deep non-REM sleep, light non-REM sleep, and REM sleep, and the sleep state determining unit 144 determines which of the levels the depth of sleep corresponds to.

The sleep controlling unit 150 determines the timing of applying a stimulus to the subject in accordance with the sleep state. The stimulus applying unit 152 applies a stimulus of a certain intensity to the subject at the timing determined by the sleep controlling unit 150. The stimulus applying unit 152 may be a loudspeaker. In this case, the stimulus applying unit 152 produces a sound of a certain volume level. Otherwise, the stimulus applying unit 152 may be configured to use vibration, light, smell, oxygen, electricity, or a combination of any of these for the stimulus to apply to the subject.

The depth of sleep is determined by comparing the autonomic nerve indices and the threshold value value. However, in the example shown in FIG. 5, the values of the autonomic nerve indices that correspond to both REM sleep and non-REM sleep gradually increase with the passage of time. This is because of the circadian rhythm. When the bases of the autonomic nerve indices themselves increase as in this case, it is highly likely that an error is caused in the determination of the depth of sleep if all the autonomic nerve indices are incorporated.

Individual differences also affect the autonomic nerve indices. For this reason, the use of all the autonomic nerve indices between 11 p.m. and 5 a.m. may prevent the sleep state from being accurately judged.

According to the embodiment, the sleep state determining unit 144 makes a judgment on the sleep state at each time within a cycle frame by using the autonomic nerve indices in the cycle frame only. This avoids the influence of the circadian rhythm and the like, and thereby improves the accuracy of sleep state judgment.

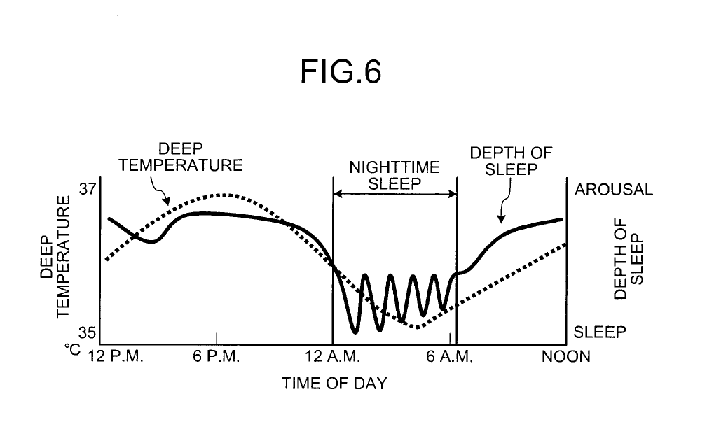

A living body has a circadian rhythm in a cycle of a day. In other words, the body temperature in a deep portion, which is the temperature inside the body, mildly changes in a cycle of a day. As shown in FIG. 6, the body temperature in a deep portion has its minimal point during the sleep and increases as the time of rising nears. Furthermore, as shown in FIG. 6, the sleep in the small hours between midnight and 6 a.m. has a sleep rhythm so that the depth of sleep changes in a cycle of approximately 90 minutes. The sleep in the small hours is a non-REM sleep that requires sound sleep.

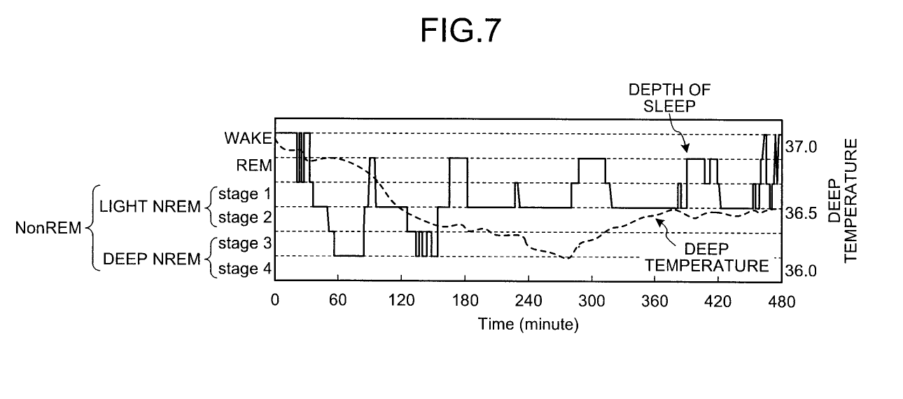

In general, during the sleep at night, the body temperature in a deep portion that serves as an indicator of the circadian rhythm is lowered right after falling asleep. Then, the temperature reaches its minimal value during the sleep and goes up as the time of rising approaches. As indicated in FIG. 7, when the body temperature in a deep portion is decreasing during the sleep, REM sleep, light non-REM sleep, and then deep non-REM sleep are detected. On the other hand, during the hours when the body temperature in a deep portion is going up after it reaches the minimal value, deep non-REM sleep is no longer detected, but light non-REM sleep and REM sleep are detected.

In the sleep state, deep non-REM sleep is regarded as sleep of the brain. During this sleep, the brain is resting. REM sleep is regarded as sleep of the body. During this sleep, the body is resting. Light non-REM sleep has a depth of sleep between the two sleep states. Light non-REM sleep can be considered as the state in which the brain is trying to decide whether to rest more or wake up. As a matter of fact, at the time when the body temperature in a deep portion takes on the minimal value, the subject is most likely in light non-REM sleep. It is considered that, when the brain takes sufficient sleep and thus decides to lead the body to arousal, the brain changes the circadian rhythm to bring the body temperature in a deep portion to an upward tendency.

The deep non-REM sleep corresponds to the sleep stages 3 and 4 of the four stages generally used for the depth of sleep. The light non-REM sleep corresponds to the sleep stages 1 and 2.

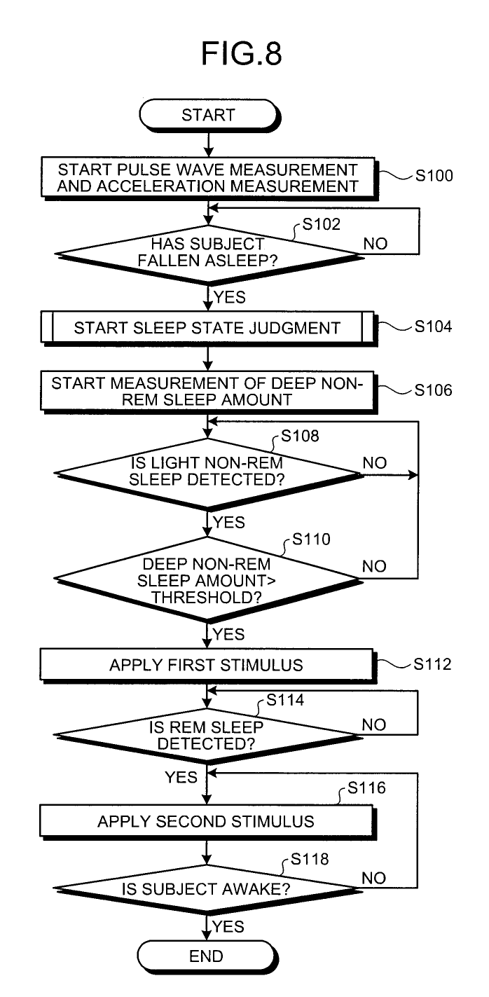

For the purpose of the subject's refreshed awakening, the sleep controlling system 1 according to the embodiment performs a process to actively produce the temperature and sleep state as indicated in FIGS. 6 and 7. For instance, when the input unit 102 receives an instruction for start from the subject, the sleep controlling system 1 begins the sleep controlling process as shown in FIG. 8. Otherwise, the sleep controlling system 1 may be configured to begin the sleep controlling process at a preset time. In this case, the subject inputs the start time from the input unit 102 in advance.

In the sleep controlling process, first, the pulse wave measuring unit 124 starts pulse wave measurement, and the acceleration measuring unit 122 starts acceleration measurement (step S100). The arousal determining unit 138 determines whether the subject is in the arousal or sleep state, based on the amount of body movement. When the arousal determining unit 138 determines that the subject is in the sleep state, or in other words when the hypnagogic state is detected (Yes at step S102), the sleep state determining unit 144 starts the sleep state judgment (step S104). Further, the sleep controlling unit 150 starts the measurement of the deep non-REM sleep amount (step S106). More specifically, the sleep controlling unit 150 starts counting the total length of time of taking deep non-REM sleep as the deep non-REM sleep amount. For instance, a counter is arranged in the storage unit 106 so that the sleep controlling unit 150 increments the counter each time of detection of deep non-REM sleep.

When the sleep state determining unit 144 detects light non-REM sleep (Yes at step S108), the sleep controlling unit 150 compares the amount of, or in other words, the total length of time of, deep non-REM sleep with a predetermined threshold value. The threshold value is a length of time after which the subject feels that he/she has had enough sleep. The threshold value is input by the subject in advance and stored in the storage unit 106.

When the total length of time of deep non-REM sleep is greater than the threshold value (Yes at step S110), the sleep controlling unit 150 determines that the subject has had sufficient deep non-REM sleep and sends an instruction to the stimulus applying unit 152 to apply a first stimulus to the subject. In response, the stimulus applying unit 152 applies the first stimulus to the subject (step S112). The intensity of the first stimulus should be lower than a predetermined threshold value.

When the total length of time of taking the deep non-REM sleep exceeds the threshold value, it is determined that the body temperature in a deep portion indicated in FIG. 7 has passed the point of the minimal value. When the body temperature in a deep portion has passed the minimal value, the stimulus applying unit 152 applies the first stimulus to the subject so that the sleep would not go back to the deep non-REM sleep state. From this standpoint, it is preferable that the first stimulus be set to a suitable intensity to prevent the subject from falling into the deep non-REM sleep but not too high to wake the subject up.

In addition, the sleep controlling system 1 may be configured to determine the threshold value that is to be compared with the total length of time of taking the deep non-REM sleep based on the measurement results on the subject in the past, instead of using the input from the subject. More specifically, the length of time of taking the deep non-REM sleep is stored in the storage unit 106 during the sleep. Then, the sleep controlling system 1 receives, from the subject, input of information indicating whether the subject feels refreshed on awakening, and determines a threshold value from the length of time with which the subject feels refreshed. Moreover, the sleep controlling system 1 may conduct the measurement for several times and thereby find the average and minimal values for the total length of time. The sleep controlling system 1 may determine the threshold value for each subject, or adopt a general value as the threshold value, regardless of each individual subject.

When the total length of time of deep non-REM sleep is equal to or below the threshold value at step S110 (No at step S110), the system goes back to step S108. This is because the body temperature in a deep portion has not reached the minimal value and thus it is considered that the amount of deep non-REM sleep is insufficient.

After applying the first stimulus (step S112), the sleep controlling unit 150 conducts REM sleep detection. When REM sleep is detected (Yes at step S114), the sleep controlling unit 150 sends an instruction to the stimulus applying unit 152 to apply a second stimulus. In response, the stimulus applying unit 152 applies the second stimulus to the subject (step S116). When the subject wakes up (Yes at step S118), the stimulus applying unit 152 stops applying the second stimulus, and the sleep control process is completed.

The second stimulus is applied to wake the subject up. Thus, the second stimulus should be set to a higher intensity than that of the first stimulus. It is preferable that the intensities of the first and second stimuli be set to suitable levels for the subject in advance by experimentally giving the subject a stimulus of a certain intensity during sleep. The intensities may be set for each individual or set to a common value obtained from an average of multiple subjects.

It is known that REM sleep is a state in which the depth of sleep is the smallest, and thus that people having REM sleep can easily wake up. Thus, the subject can be woken up and feel refreshed by conducting a waking operation during the REM sleep.

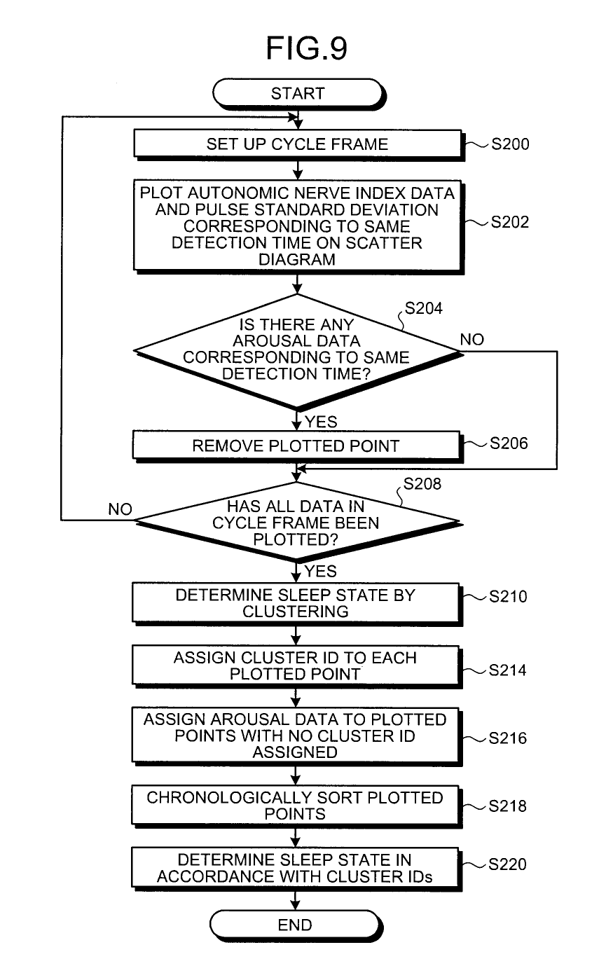

In the sleep state determining process started at step S104 in FIG. 8, first, the cycle frame setting unit 140 sets up a cycle frame, as indicated in FIG. 9 (step S200). According to the embodiment, the cycle frame is set to 120 minutes. Thus, the processing from steps S202 through S206 is executed onto the data equivalent to 120 minutes.

Then, the sleep state determining unit 144 plots the autonomic nerve index data stored in the storage unit 106 in association with the detection time and the pulse deviation stored in the storage unit 106 in association with the same detection time on plane coordinates of a scatter diagram (step S202). When arousal data is stored in the storage unit 106 in association with the detection time that corresponds to the plotted detection time (Yes at step S204), the sleep state determining unit 144 removes the corresponding plotted points from the scatter diagram (step S206). The sleep state determining unit 144 can thereby determine the sleep state based on the data obtained during the sleep only. Hence, the determination of the sleep state can be made with high accuracy.

The x coordinate on the plane coordinates indicates LF/HF, which is the autonomic nerve index, and the y coordinate indicates the pulse deviation. Otherwise, the sleep state determining unit 144 may be configured to plot the autonomic nerve index LF on the x coordinate and HF on the y coordinate.

The processing from steps S202 through S206 is repeated until all the data in the cycle frame is plotted on the scatter diagram (Yes at step S208). According to the embodiment, the processing is repeated until the 120 items of data are plotted on the scatter diagram.

Next, the sleep state determining unit 144 clusters the plotted points on the scatter diagram to determine the sleep state (step S210). More specifically, the sleep state determining unit 144 first divides the plotted points into three clusters by use of the k-means algorithm. The cluster whose center is the closest to the origin point is referred to as the first cluster, the cluster the second closest thereto is the second cluster, and the furthest cluster is the third cluster. According to the embodiment, the k-means algorithm is incorporated as a clustering method from a standpoint of reducing the data processing load, but the invention is not limited thereto. Other examples of clustering methods include the FCM method and the entropy method.

Then, the sleep state determining unit 144 provides each item of data that is plotted on the scatter diagram with a cluster ID (step S214). At this point, when there is an item of data without a cluster ID, the item is provided with a cluster ID that indicates arousal data (step S216). Next, the sleep state determining unit 144 sorts the cluster ID-added data items in a chronological manner (step S218).

Thereafter, the sleep state determining unit 144 determines the sleep state based on the cluster IDs added to the items of data that are chronologically sorted (step S220). In particular, the sleep state determining unit 144 determines the sleep state of the detection time that corresponds to the first cluster as deep non-REM sleep. The sleep state determining unit 144 determines the sleep states of the detection times that correspond to the second and third clusters as light non-REM sleep and REM sleep, respectively. The sleep state determination can be conducted with high accuracy by the clustering operation of the sleep state determining unit 144 as described above.

When a 120-minute cycle frame is set up for every minute as in the above description, a time point is included in different cycle frames. Moreover, the determination result on this time may be different among the cycle frames. From the standpoint of real-time data acquisition, the sleep state determining unit 144 should make a determination in accordance with a process using a cycle frame in which the target time is positioned close to the latest end of the cycle frame. In other words, the sleep state determining unit 144 should determine the sleep state at the target time after a certain length of time passes, in accordance with a process performed on a cycle frame that includes the target time at the latest end thereof.

According to the embodiment, the sleep state determining unit 144 adopts the clustering method for the sleep state determination, but the invention is not limited thereto. In other words, any method can be adopted as long as the sleep state determining unit 144 can determine which of the deep non-REM sleep, light non-REM sleep, and the REM sleep the subject is in. For example, the sleep state determining unit 144 may compare the autonomic nerve index with a predetermined threshold value to determine which of the deep non-REM sleep, light non-REM sleep, and REM sleep the sleep state is.

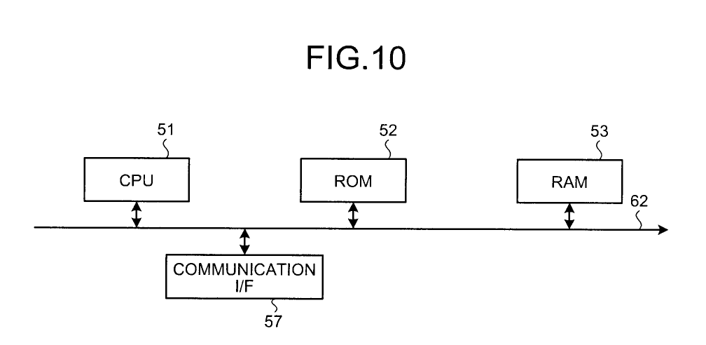

As illustrated in FIG. 10, the main body 10 includes, as a hardware structure, a ROM 52 that stores therein a sleep controlling program for which the main body 10 executes a sleep controlling process and the like, a CPU 51 that controls the units of the main body 10 in accordance with the program stored in the ROM 52, a RAM 53 that stores therein various kinds of data necessary to control the main body 10, a communication interface 57 connected to a network to conduct communications, and a bus 62 that connect these components.

The sleep controlling program of the main body 10 may be stored and offered in a computer-readable recording medium such as a CD-ROM, floppy disk (trademark, FD), DVD and the like as a file of an installable or executable format.

If this is the case, the sleep controlling program is read from the recording medium and executed by the main body 10 so that the program is loaded on the main storage device and the units explained above as the software structure are created on the main storage device.

Otherwise, the sleep controlling program according to the embodiment may be configured to be stored in a computer connected to a network such as the Internet and provided by downloading via the network.

The present invention has been explained by using the embodiment. Various changes and modifications may be added to the embodiment.

In a first modification example, instead of measuring the length of time of taking deep non-REM sleep and thereby determining whether the body temperature in a deep portion takes on the minimal value as shown in FIG. 7, the sleep controlling system 1 may be configured to count the number of events in which the subject falls into deep non-REM sleep.

In a second modification example, the sleep controlling system 1 may be configured to control an air-conditioning system installed in the room where the subject is lying. More specifically, at step S110 in FIG. 8, the room temperature may be lowered until the subject takes a sufficient amount of deep non-REM sleep to create an environment in which the subject can easily take deep non-REM sleep.

Furthermore, as a third modification example, the sleep controlling system 1 may be configured to determine the sleep state by use of a polysomnogram instead of pulse wave measurement. If this is the case, the sleep controlling system 1 differentiates the sleep stages 1 to 4, and determines the sleep stages 1 and 2 as light non-REM sleep, and the sleep stages 3 and 4 as deep non-REM sleep. The sleep controlling system 1 will do as long as it is capable of performing control in accordance with the depth of sleep, and thus the indices for calculating the depth of sleep are not limited to the ones described in the embodiment.

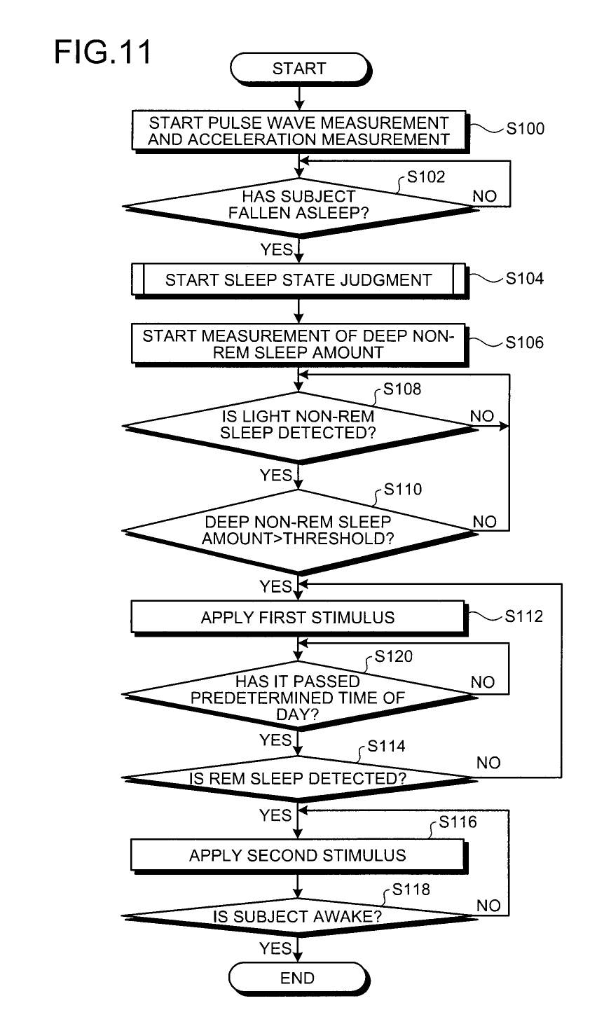

According to a second embodiment, the sleep controlling system 1 applies a first stimulus to the subject, and when it is past a specified time of day (Yes at step S120), the sleep controlling system 1 starts detection of REM sleep, as indicated in FIG. 11. For instance, when the input unit 102 obtains the desired rising time from the subject and the storage unit 106 stores therein the desired rising time, the specified time is determined as a predetermined number of minutes before the desired rising time. For instance, the specified time may be defined as 90 minutes before the desired rising time. The sleep controlling system 1 can thereby wake the subject up at a time of day close to the desired rising time and also during the REM sleep state. Hence, the subject can get up refreshed at a desired time. The specified time should be in a certain range of time with reference to the desired rising time, and may be set within 30 minutes before or after the desired rising time.

The rest of the structure and the process of the sleep controlling system 1 according to the second embodiment is the same as those of the sleep controlling system 1 according to the first embodiment.

In the sleep controlling system 1 according to this embodiment, the desired rising time is input by the subject on the input unit 102, but the sleep controlling system 1 may be configured to obtain the desired sleep hours from the input unit 102 and stores the obtained desired sleep hours in the storage unit 106. In this case, the desired rising time may be calculated by adding the desired sleep hours to the time of falling asleep, and the specified time may be a predetermined number of minutes before this desired rising time.

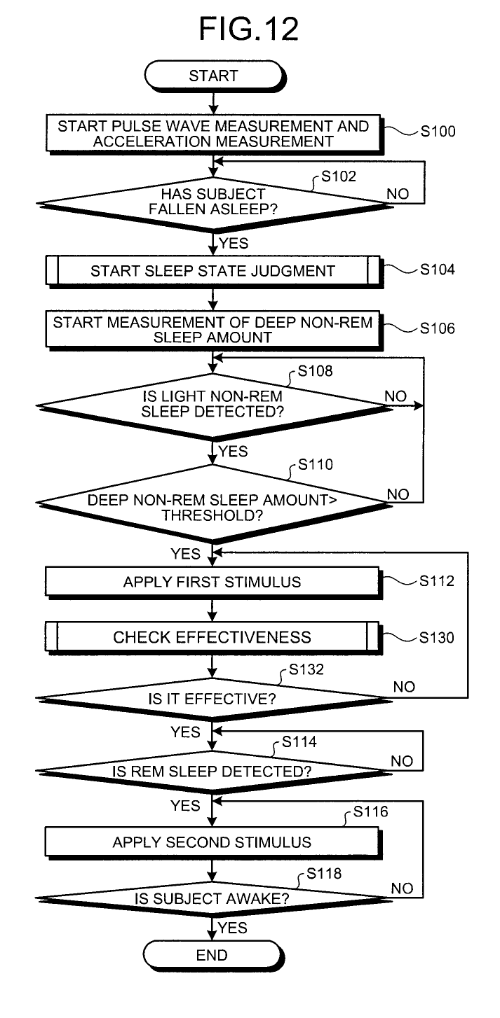

In the sleep controlling system 1 according to the third embodiment, after the stimulus applying unit 152 applies the first stimulus to the subject (step S112), the sleep controlling unit 150 determines whether the first stimulus is effective based on the amount of body movement (step S130), as indicated in FIG. 12. More specifically, the sleep controlling unit 150 stores the time to at which the first stimulus is applied in the storage unit 106 (step S140), as indicated in FIG. 13. If there is any body movement (Yes at step S142), the sleep controlling unit 150 determines that the first stimulus is effective (step S144). On the other hand, if there is no body movement (No at step S142), and if the current time t satisfies the expression (1) (Yes at step S144), the sleep controlling unit 150 determines that the first stimulus is ineffective (step S148). In the expression (1), Δt represents a predetermined time. At is a presumed length of time between the occurrence of a K-complex and the occurrence of body movement and may be set to 10 seconds.

t−t₀>Δt (1)

On the other hand, when t does not satisfy the expression (1) at step S146 (No at step S146), the system returns to step S142.

According to the third embodiment, the sleep controlling system 1 not only applies the first stimulus to the subject but also checks the effectiveness of the first stimulus to the subject. Thus, the sleep controlling system 1 can reliably bring the sleep state from the deep non-REM sleep to the light non-REM sleep and REM sleep.

The rest of the structure and the process of the sleep controlling system 1 according to the third embodiment is the same as those of the sleep controlling system 1 according to other embodiments.

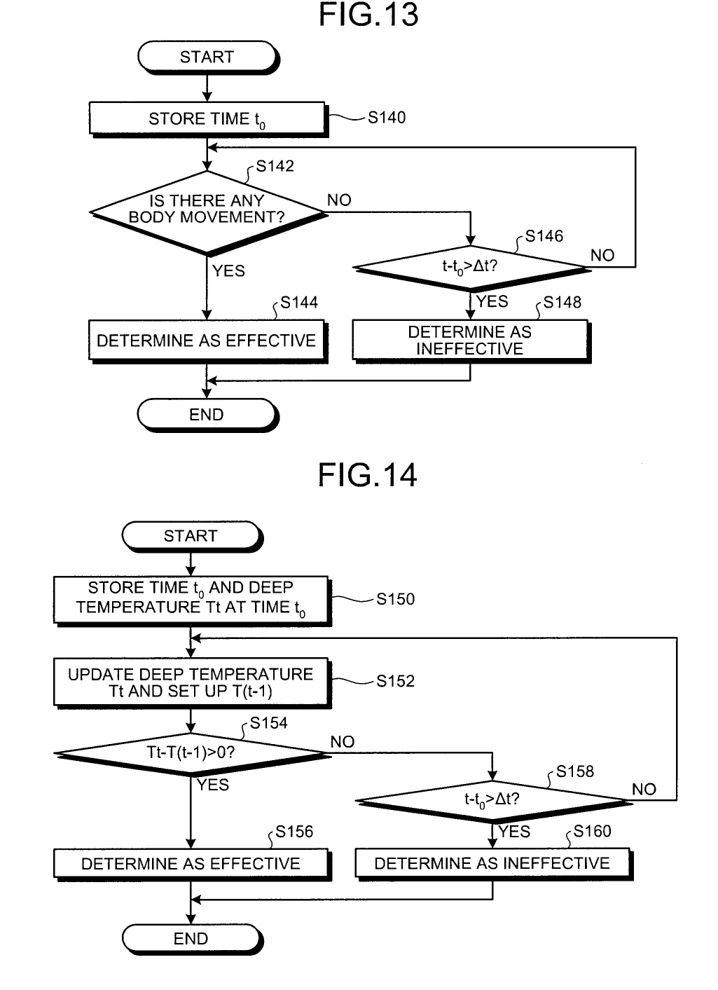

The sleep controlling system 1 according to the fourth embodiment also checks the effectiveness of the first stimulus in the similar manner to the sleep controlling system 1 according to the third embodiment, but the effectiveness is checked based on the body temperature in a deep portion. More specifically, in the sleep controlling system 1 according to the fourth embodiment, after the stimulus applying unit 152 applies the first stimulus to the subject (step S120), the sleep controlling unit 150 checks the effectiveness of the first stimulus. In particular, as indicated in FIG. 14, the time to of applying the first stimulus and the body temperature in a deep portion Tt of the subject at the time to are stored in the storage unit 106 (step S150). Next, the sleep controlling unit 150 updates the body temperature in a deep portion Tt to the body temperature in a deep portion at the current time t. Furthermore, the sleep controlling unit 150 sets the body temperature in a deep portion at a time a predetermined number of minutes before the current time t to T(t−1) (step S152).

When Tt satisfies the expression (2), or in other words, when the differential element of the body temperature in a deep portion takes on a positive value (Yes at step S154), the sleep controlling unit 150 determines that the first stimulus is effective (step S156).

Tt−T(t−1)>0 (2)

On the other hand, when Tt does not satisfy the expression (2) at step S154 (No at step S154) but t satisfies the expression (1) (Yes at step S160), the sleep controlling unit 150 determines that the first stimulus is ineffective (step S160). When t does not satisfy the expression (1) at step S158 (Yes at step S158), the system goes back to step S152.

The sleep controlling system 1 according to the fourth embodiment checks the effectiveness of the first stimulus to the subject in the same manner as the sleep controlling system 1 according to the third embodiment. Thus, the sleep controlling system 1 can reliably leads the sleep state from the deep non-REM sleep to the light non-REM sleep and REM sleep.

The rest of the structure and the process of the sleep controlling system 1 according to the fourth embodiment is the same as those of the sleep controlling system 1 according to other embodiments.

The sleep controlling system 1 according to the fourth embodiment determines the effectiveness of the first stimulus with reference to the differential element of the body temperature in a deep portion Tt at the current time t and the body temperature in a deep portion T(t−1) at a time a predetermined number of minutes before the current time, but the determination on the effectiveness of the first stimulus is not limited thereto. For instance, the sleep controlling system 1 may be configured to determine the effectiveness of the first stimulus with reference to the differential element of the body temperature in a deep portion at the current time t and the body temperature in a deep portion at the time t₀ of applying the first stimulus. In particular, the sleep controlling unit 150 may be configured in such a manner that the first stimulus is determined as being effective when the result of subtracting the body temperature in a deep portion at the time t₀ from the body temperature in a deep portion at the current time t is larger than 0, while the first stimulus is determined as being ineffective when the result is equal to or smaller than 0.

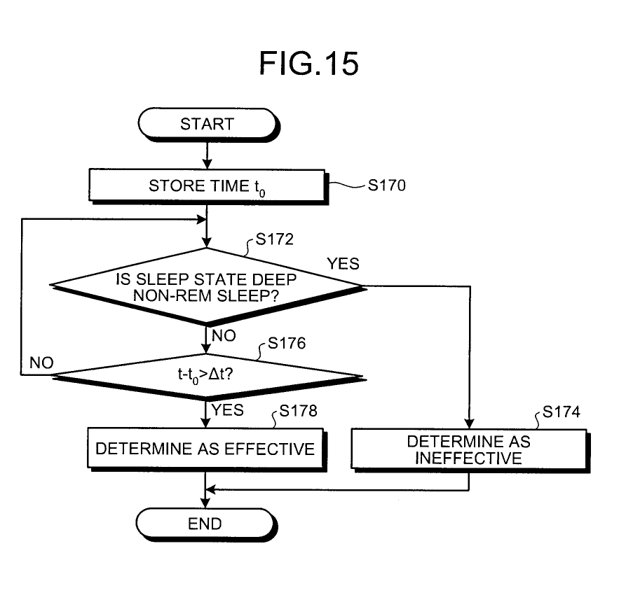

The sleep controlling system 1 according to a fifth embodiment also checks the effectiveness of the first stimulus in the same manner as the sleep controlling system 1 according to the third and fourth embodiments. However, the effectiveness of the first stimulus is determined with reference to the sleep state. More specifically, after the stimulus applying unit 152 applies the first stimulus to the subject (step S120), the sleep controlling unit 150 checks the effectiveness of the first stimulus. That is, as indicated in FIG. 15, the time to at which the stimulus applying unit 152 applies the first stimulus is stored in the storage unit 106 (step S170). Next, the sleep controlling unit 150 finds whether the determination result of the sleep state determining unit 144 is deep non-REM sleep. When the deep non-REM sleep is detected (Yes at step S172), the sleep controlling unit 150 determines that the first stimulus is ineffective (step S174). In other words, if deep non-REM sleep is detected after the first stimulus is applied, the first stimulus is found out to be ineffective.

On the other hand, when deep non-REM sleep is not detected, or in other words, when either light non-REM sleep or REM sleep is detected, or when arousal is detected (No at step S172), the sleep controlling unit 150 further determines whether the current time t satisfies the relationship of the expression (1), or in other words whether a predetermined number of minutes (Δt) elapse after the first stimulus.

When the current time t satisfies the expression (1) (Yes at step S176), the sleep controlling unit 150 determines that the first stimulus is effective (step S178). When a certain period of time elapses without falling into the deep non-REM sleep, the sleep controlling unit 150 determines that the first stimulus is effective. If the current time t does not satisfy the expression (1) (No at step S176), the system goes back to step S172, where the sleep controlling unit 150 checks the determination result of the sleep state.

The rest of the structure and the process of the sleep controlling system 1 according to the fifth embodiment is the same as those of the sleep controlling system 1 according to other embodiments.

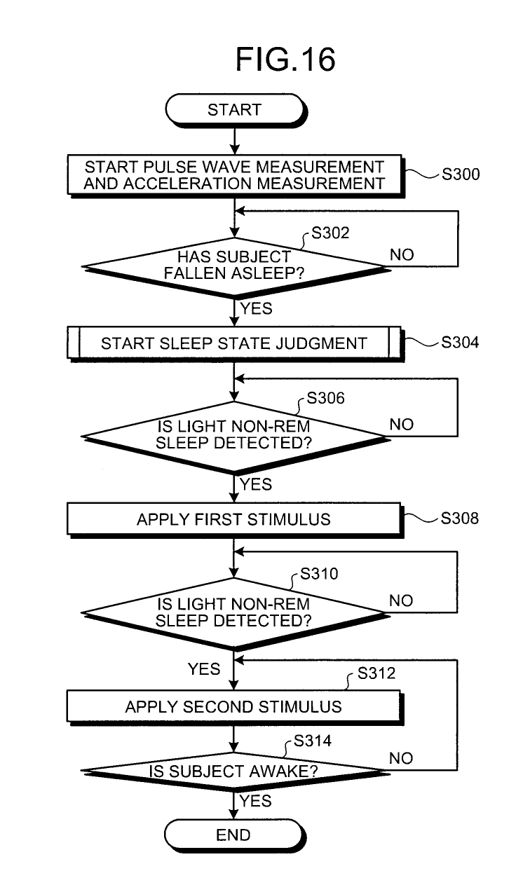

The sleep controlling system 1 according to a sixth embodiment performs control of sleep during the daytime, or in other words control of napping. To wake refreshed from a nap, it is preferable that one have sleep before falling into the deep non-REM sleep state. As indicated in FIG. 16, the sleep controlling system 1 first starts the measurement of the pulse wave and acceleration (step S300), and when it is determined that the subject falls asleep (Yes at step S302), the sleep controlling system 1 begins the judgment on the sleep state (step S304). The above process is the same as the process from steps S100 to S104 according to the first embodiment.

The sleep controlling unit 150 does not measure the amount of deep non-REM sleep, but detects light non-REM sleep. When light non-REM sleep is detected (Yes at step S306), the stimulus applying unit 152 applies the first stimulus to the subject (step S308). This prevents the subject from falling into deep non-REM sleep.

When the sleep controlling unit 150 detects light non-REM sleep after the application of the first stimulus (Yes at step S310), the stimulus applying unit 152 applies the second stimulus to the subject (step S312). If the subject wakes up (Yes at step S314), the stimulus applying unit 152 stops applying the second stimulus, and the sleep controlling process is terminated. During a daytime sleep, the first stimulus deep is applied so that the subject would not fall into deep non-REM sleep and would maintain the light non-REM sleep. When the subject does not fall into deep non-REM sleep, the sleep tends to go deeper but would rarely stay in the REM sleep state. For this reason, the timing of applying the second stimulus should be during light non-REM sleep, unlike in the situation of night sleep.

The rest of the structure and the process of the sleep controlling system 1 according to the sixth embodiment is the same as those of the sleep controlling system 1 according to other embodiments.

According to this embodiment, the time of rising and hours of sleep may be preset in a similar manner to the sleep controlling system 1 according to the second embodiment. If this is the case, the sleep controlling unit 150 may start detecting the light non-REM sleep (step S310) to determine the timing of applying the second stimulus after it is past a specified time of day that is configured in accordance with the rising time or sleep hours.

In a similar manner to the sleep controlling system 1 according to the third to fifth embodiments, the effectiveness of the first stimulus may be checked after the first stimulus is applied.

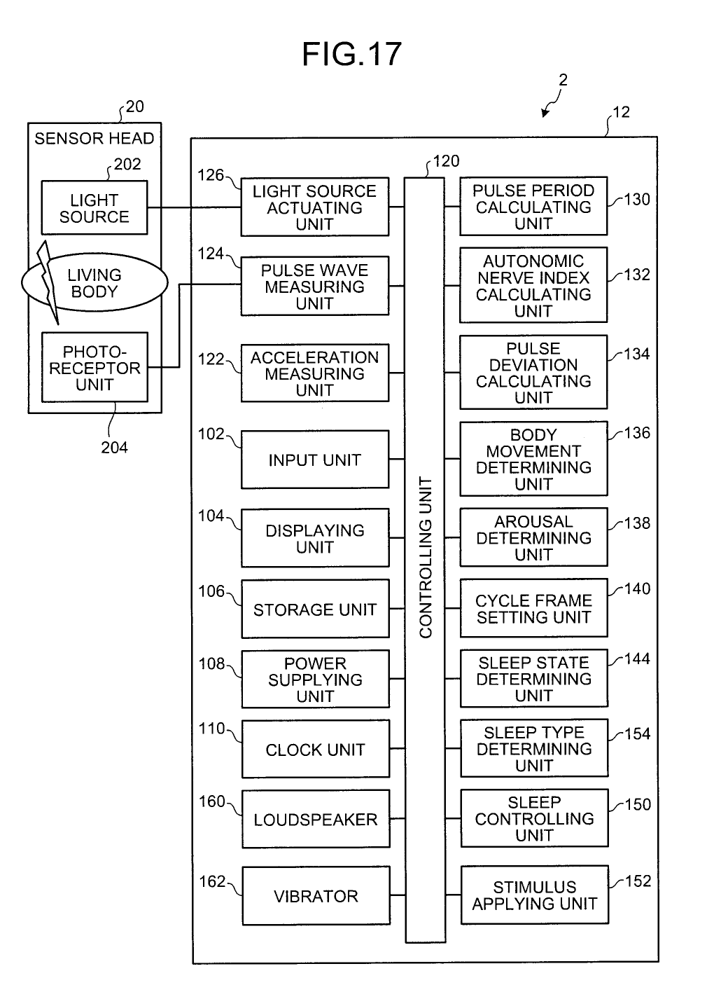

According to a seventh embodiment, a main body 12 of a sleep controlling system 2 includes a sleep type determining unit 154 in addition to the functional structure of the main body 10 according to other embodiments, as illustrated in FIG. 17. The sleep controlling system 2 according to the seventh embodiment is capable of conducting sleep control during the daytime and nighttime.

The sleep type determining unit 154 determines the type of sleep. There are two types of sleep, daytime sleep and nighttime sleep. The subject inputs daytime sleep or nighttime sleep into the input unit 102, and the sleep type determining unit 154 determines the type of sleep based on this input information. In this manner, the subject is allowed to designate the nighttime sleep control in which the sleep is controlled so that the subject can go into deep non-REM sleep for sufficient resting. Or if the subject wants to take a nap, daytime sleep control can be designated so that the subject can wake up refreshed after a relatively short time of resting, before going into deep non-REM sleep.

Moreover, the sleep type determining unit 154 may be configured to determine the type of sleep in accordance with the time of day at which the input unit 102 receives the instruction for starting the sleep controlling process. For instance, when an instruction of start is received between 8 p.m. and 8 a.m., the sleep type determining unit 154 determines that the type of sleep is nighttime sleep. For an instruction of start received at any other time, the sleep type determining unit 154 determines that the type of sleep is daytime sleep. Furthermore, a span of time for the night sleep may be predetermined, for example, by the subject.

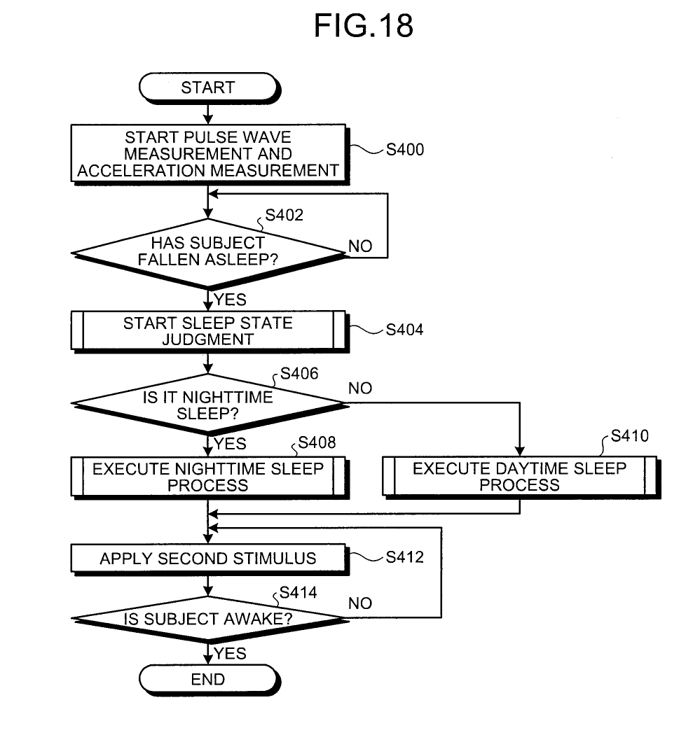

As indicated in FIG. 18, the sleep controlling system 2 according to the seventh embodiment first begins the measurement of the pulse wave and acceleration in the sleep controlling process (step S400). Next, when it is determined that the subject falls asleep (Yes at step S402), the sleep controlling system 2 begins the judgment of the sleep state (step S404). The process up to this step is the same as the process between steps S100 and S104 of the sleep controlling system 1 according to the first embodiment.

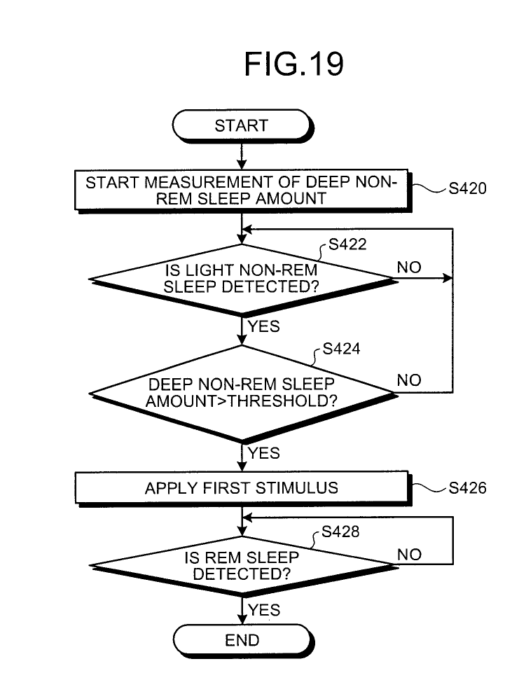

Next, the sleep type determining unit 154 determines the type of sleep. If the type of sleep is nighttime sleep (Yes at step S406), the sleep controlling unit 150 and the stimulus applying unit 152 perform a process for nighttime sleep (step S408). The details of the process for nighttime sleep shown in FIG. 19 (step S408) are the same as the process from steps S106 through S114 according to the first embodiment.

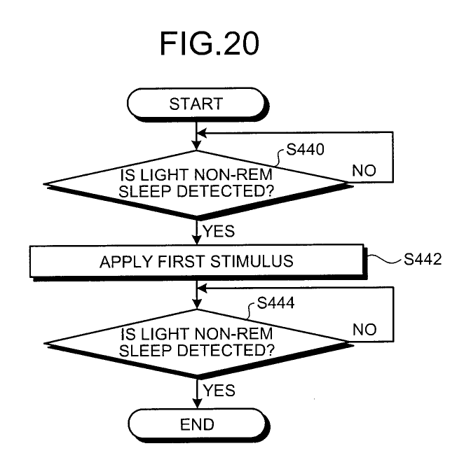

On the other hand, when the type of sleep is daytime sleep (No at step S406), the sleep controlling unit 150 and the stimulus applying unit 152 perform a process for daytime sleep (step S410). The details of the process for daytime sleep shown in FIG. 20 (step S410) are the same as the process from steps S306 through S310 according to the sixth embodiment.

When the timing of applying the second stimulus is determined in the nighttime sleep controlling process (step S408) or the daytime sleep controlling process (step S410), the stimulus applying unit 152 applies the second stimulus to the subject (step S412) in accordance with an instruction from the sleep controlling unit 150. When the subject wakes up (Yes at step S414), the sleep controlling process is terminated.

The sleep controlling system 2 according to this embodiment determines the type of sleep and conducts sleep control in accordance with the type.

The rest of the structure and the process of the sleep controlling system 2 according to the seventh embodiment is the same as those of the sleep controlling system 1 according to other embodiments.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A sleep controlling apparatus comprising:
a measuring unit that measures biological information of a subject;
a first detecting unit that detects a sleeping state of the subject selected from a group consisting of a falling asleep state, a REM sleep state, a light non-REM sleep state and a deep non-REM sleep state, based on the biological information measured;
a first stimulating unit that applies a first stimulus of an intensity lower than a predetermined threshold value to the subject when the light non-REM sleep state is detected; and
a second stimulating unit that applies a second stimulus of an intensity higher than the first stimulus after the first stimulus is applied to the subject.

2. The apparatus according to claim 1, wherein
the first detecting unit detects a sleeping state of the subject after the first stimulus is applied to the subject; and
the second stimulating unit applies the second stimulus to the subject when the REM sleep state is detected.

3. The apparatus according to claim 1, further comprising a clock unit that counts elapsed time after the subject falls asleep, wherein
the second stimulating unit applies the second stimulus to the subject after the clock unit counts a predetermined length of elapsed time and the first stimulating unit applies the first stimulus.

4. The apparatus according to claim 3, further comprising a storage unit that stores a desired rising time of the subject, wherein
the second stimulating unit applies the second stimulus to the subject after the clock unit counts the predetermined length of time within a predetermined time frame with reference to the desired rising time.

5. The apparatus according to claim 1, wherein the first stimulating unit applies the first stimulus to the subject when the first detecting unit detects the deep non-REM sleep state and also detects the light non-REM sleep state after the deep non-REM sleep state.

6. The apparatus according to claim 5, wherein the first stimulating unit applies the first stimulus to the subject when the deep non-REM sleep state is detected for a period of time equal to or longer than a predetermined length of time and when the light non-REM sleep state is detected after the deep non-REM sleep state.

7. The apparatus according to claim 5, wherein the first stimulating unit applies the first stimulus to the subject when the deep non-REM sleep state is detected for a number of times equal to or greater than a predetermined number and when the light non-REM sleep state is detected after the deep non-REM sleep state.

8. The apparatus according to claim 1, further comprising a second detecting unit that detects body movement of the subject after the first stimulus is applied, wherein
the first stimulating unit applies the first stimulus again to the subject when the body movement is not detected.

9. The apparatus according to claim 8, wherein the second detecting unit detects the body movement of the subject at a predetermined length of time elapses after the first stimulus is applied.

10. The apparatus according to claim 1, further comprising a third detecting unit that detects a body temperature in a deep portion of the subject after the first stimulus is applied, wherein
the first stimulating unit applies the first stimulus again to the subject when the body temperature in a deep portion falls at any time after the first stimulus is applied until a predetermined length of time elapses thereafter.

11. The apparatus according to claim 1, wherein the first stimulating unit applies the first stimulus again to the subject when the deep non-REM sleep state is detected at any time after the first stimulus is applied until a predetermined length of time elapses thereafter.

12. The apparatus according to claim 1, wherein the second stimulating unit applies the second stimulus to the subject when the light non-REM sleep state is detected after the first stimulus is applied.

13. The apparatus according to claim 1, further comprising a designation receiving unit that receives from the subject designation of either one of a first sleep in which the deep non-REM sleep state is included and a second sleep in which the deep non-REM sleep state is not included, wherein when the designation receiving unit receives the designation of the first sleep, the second stimulating unit applies the second stimulus to the subject when the REM sleep state is detected after the first stimulus is applied.

14. The apparatus according to claim 1, further comprising a designation receiving unit that receives from the subject designation of either one of a first sleep in which the deep non-REM sleep state is included and a second sleep in which the deep non-REM sleep state is not included, wherein when the designation receiving unit receives the designation of the second sleep, the second stimulating unit applies the second stimulus to the subject when the light non-REM sleep state is detected after the first stimulus is applied.

15. A sleep controlling method comprising:
measuring biological information of a subject;
detecting a sleeping state of the subject selected from the group consisting of a falling asleep state, a REM sleep state, a light non-REM sleep state and a deep non-REM sleep state, based on the biological information;
applying a first stimulus of an intensity lower than a predetermined threshold value to the subject when the light non-REM sleep state is detected; and
applying a second stimulus of an intensity higher than the first stimulus after the first stimulus is applied to the subject.

16. The method according to claim 15, further comprising:
detecting a sleeping state of the subject after the first stimulus is applied to the subject; and
applying the second stimulus to the subject when the REM sleep state is detected.

17. The method according to claim 15, further comprising:
applying the first stimulus to the subject when the deep non-REM sleep state is detected and the light non-REM sleep state after the deep non-REM sleep state is also detected.

18. A computer program product having a computer readable medium including programmed instructions for performing sleep control, wherein the instructions, when executed by a computer, cause the computer to perform:
measuring biological information of a subject;
detecting a sleeping state of the subject selected from the group consisting of a falling asleep state, a REM sleep state, a light non-REM sleep state and a deep non-REM sleep state, based on the biological information,
applying a first stimulus of an intensity lower than a predetermined threshold value to the subject when the light non-REM sleep state is detected; and
applying a second stimulus of an intensity higher than the first stimulus after the first stimulus is applied to the subject.

19. The computer program product according to claim 18, wherein the instructions cause the computer to further perform:
detecting a sleeping state of the subject after the first stimulus is applied to the subject; and
applying the second stimulus to the subject when the REM sleep state is detected.

20. The computer program product according to claim 18, wherein the instructions cause the computer to further perform:
applying the first stimulus to the subject when the deep non-REM sleep state is detected and the light non-REM sleep state after the deep non-REM sleep state is also detected.
